SEQUENCE LISTING (3 sequences)

SEQ ID NO: 1 - DNA, 18 bp, synthetic construct
  source 1..18: mol_type = other DNA
gcttttgagcctagcgtt

SEQ ID NO: 2 - DNA, 22 bp, synthetic construct
  source 1..22: mol_type = other DNA
gccatgttgtcactgtctattg

SEQ ID NO: 3 - DNA, 13 bp, synthetic construct
  source 1..13: mol_type = other DNA
aacctgtcacctc

(19) United States
(12) Patent Application Publication
(10) Pub. No.: US 2024/0009118 A1
(43) Pub. Date: Jan. 11, 2024

(54) FOAM VACCINATION OF AVIANS

(71) Applicants: Hong Li, Newark, DE (US); Kenneth C. Elliott, Germantown, MD (US)

(72) Inventors: Hong Li, Newark, DE (US); Kenneth C. Elliott, Germantown, MD (US)

(73) Assignee: University of Delaware, Newark, DE (US)

(21) Appl. No.: 18/350,357

(22) Filed: Jul. 11, 2023

Related U.S. Application Data
(60) Provisional application No. 63/359,962, filed on Jul. 11, 2022.

(51) Int. Cl. A61K 9/00; A61K 39/215; A61K 47/42; A61K 47/36; A61P 31/14

(52) U.S. Cl. CPC A61K 9/0056; A61K 39/215; A61K 9/0048; A61K 47/42; A61K 47/36; A61P 31/14; A61K 2039/552

(57) ABSTRACT

The invention provides a method for vaccinating one or more avians in need thereof. The vaccination method comprises administering to the one or more avians a stable foam in an amount effective for inducing a neutralizing immune response against an infectious pathogen in the one or more avians. The foam comprises a gas and a liquid, and the liquid comprises a vaccine and a foaming agent. The avian may be a chick. Also provided is a method for preparing the stable foam.

### REFERENCE TO U.S. GOVERNMENT SUPPORT

This invention was made with government support under Grant No. 2018-67021-28106 from the U.S. Department of Agriculture. The United States has certain rights in the invention.

### CROSS-REFERENCE TO RELATED APPLICATION

This application claims priority to U.S. Provisional Application No. 63/359,962, filed Jul. 11, 2022, and the contents of which are incorporated herein by reference in their entireties for all purposes.

### REFERENCE TO SEQUENCE LISTING

The present application is being filed along with a Sequence Listing in electronic format. The Sequence Listing is provided as a file entitled UOD-562US_SequenceListing.XML created on Jul. 11, 2023, which is 3.53 KB (3,618 bytes) in size.

### FIELD OF THE INVENTION

This invention relates generally to a foam vaccination system for avians, for example, chicks in a hatchery.

### BACKGROUND OF THE INVENTION

Several methods are currently available for mass vaccination of avians. For example, eye drop vaccination is performed by applying a small amount of vaccine, approximately 0.03 ml, to an individual bird's eyes using a dropper or syringe. This vaccination technique is often used in experiments for challenge and vaccine controls due to its effectiveness with the typical titer varying from 10^{4.5}-10⁶. Vaccines for infectious bursal disease virus (IBDV), infectious bronchitis virus (IBV), infectious laryngotracheitis (ILT), and new castles disease virus (NDV) have been tested for eye drop vaccination. The effectiveness, accuracy, and uniform immunity of eye drop vaccination make it a valuable field technique. However, it is a labor-intensive technique that requires the handling of each individual.

Spray vaccination is a method of mass vaccination used to vaccinate day-old chicks against IBV and NDV. Despite advances over 40 years, spray technology can still suffer from a lack of uniform vaccine application to chicks under manufacturer recommended application.

Gel vaccination is a method of mass vaccination that utilizes gel droplets to vaccinate day-old-chicks primarily for coccidiosis. Oocysts are suspended in gel, which is then applied as small droplets onto chicks through a system of syringes suspended over the chicks. The chicks ingest these droplets, through preening. Factors such as the quantity and viability of oocysts delivered, distribution of oocysts in the gel, and viscosity of the gel need to be considered for the vaccination to be successful. However, a disadvantage of this method is that the rigorous mixing process can lead to uneven distribution of the oocysts throughout the gel drop and affect the viability of the applied agents.

In ovo vaccination is vaccination that occurs between embryo day (ED) 18 and 20. The vaccination route is specific by vaccine and occurs via the amniotic or intraembryonic route. Successful in ovo vaccination makes use of live and recombinant vaccines. In ovo vaccination is used to vaccinate for Marek's Disease, and less commonly reovirus, IBDV, ILT, fowl pox, and some NDV. For successful vaccination, the age of the embryo, the site of the vaccination, the mixing of the vaccine, and sanitization of the machine are all important factors. Disadvantages include the cost requirements of the equipment and training, and the potential for bacterial and fungal contamination through the open hole in the eggshell or unclean equipment.

Subcutaneous vaccination (SC) is when vaccine is inoculated into a bird under the skin, typically at the back of the neck. The recommended dose for each chick is between 0.2-0.5 ml. A dye can be mixed with the vaccine that can be visually identified at the site of injection to confirm vaccination. Needles on the machine should be changed frequently to ensure proper vaccination of the birds. The average rate of vaccination is 1600 to 2000 chicks per hour. Subcutaneous vaccinations for ILT, NDV, and IBDV are available. SC vaccination has several disadvantages including bird stress, possible localized tissue damage, and machinery sanitation.

There remains a need for an easy, fast and uniform vaccination application to avians, especially a mass vaccination application to chicks in hatcheries.

### SUMMARY OF THE INVENTION

The present invention relates to foam vaccination of avians, for example, chicks. The inventors have surprisingly discovered a foam vaccination method for a uniform and efficient application to chicks.

The present invention provides a method for vaccinating one or more avians in need thereof. The vaccination method comprises administering to the one or more avians a stable foam in an amount effective for inducing a neutralizing immune response against an infectious pathogen in the one or more avians. The foam comprises a gas and a liquid, and wherein the liquid comprises a vaccine and a foaming agent.

According to the vaccination method, the foam may be ingestible. The vaccination method may further comprise ingesting the foam by the one or more avians.

The vaccination method may further comprise contacting the foam with an eye of the one or more avians such that the vaccine enters the one or more avians via the eye.

According to the vaccination method, the one or more avians may be one or more day-old chicks. The infectious pathogen may cause an infectious disease selected from the group consisting of Marek's disease, coccidiosis, mycoplasma gallisepticum infections, infectious bursal disease, encephalomyelitis, fowlpox, laryngotracheitis, Newcastle disease, and infectious bronchitis.

According to the vaccination method, the foaming agent may comprise a surfactant. The surfactant may comprise one or more proteins. The foaming agent may comprise a viscosity agent. The foaming agent may comprise an aqueous solvent. The foam may have an expansion rate of 20-40, a bubble size of 3-4 mm, a liquid drainage of 50-95% at 10 minutes, a deterioration rate of 95-99% at 5 minutes, or a combination thereof. The ratio of the liquid to the gas in the foam may be from 1:20 to 1:200.

The present invention also provides a method for preparing a stable vaccine foam. The preparation method comprises (a) mixing a foaming agent and a vaccine capable of inducing a neutralizing immune response against an infectious pathogen in an avian in need thereof, whereby a liquid is formed; (b) mixing the liquid with a gas, whereby a liquid gas mixture is formed; (c) forcing the liquid gas mixture through a filter, whereby a stable foam is formed.

According to the preparation method, the foaming agent may comprise a surfactant. The surfactant may comprise one or more proteins. The foaming agent may comprise a viscosity agent. The foaming agent may comprise an aqueous solvent. The foam may have an expansion rate of 20-40, a bubble size of 3-4 mm, a liquid drainage of 50-95% at 10 minutes, a deterioration rate of 95-99% at 5 minutes, or a combination thereof. The ratio of the liquid to the gas in the foam may be from 1:20 to 1:200. The foam may be ingestible.

According to the preparation method, the avian may be a day-old chick. The infectious pathogen may cause a disease selected from the group consisting of Marek's disease, coccidiosis, mycoplasma gallisepticum infections, infectious bursal disease, encephalomyelitis, fowlpox, laryngotracheitis, Newcastle disease, and infectious bronchitis.

### BRIEF DESCRIPTION OF THE DRAWINGS

FIGS. 1A-G illustrate a foam nozzle and its components: (A) Photographs of components of a foam nozzle; (B) Side view of components of a foam nozzle; (C) Foam nozzle breakdown; (D) bung for aluminum nozzle; (E) Top Bung aluminum nozzle; (F) Aluminum nozzle incorporated bung; and (G) Aluminum cove tip.

FIG. 2 shows an outline of an inline mixing chamber: (A) Exterior photo of mixing chamber; and (B) interior photo of mixing chamber.

FIG. 3 shows average liquid drainage over 10 minutes for a final nozzle using formulation 7.1.

FIG. 4 shows an average deterioration rate over 5 minutes for a final nozzle using formulation 7.1.

FIG. 5 shows the average amount of the application material ingested by chicks. The foam application material was formulation 7.1. The spray application material was DI H₂O.

FIGS. 6A-D show (A) an outline a full system; (B) full system components: air manifold, conveyor belt, foam manifold, 80/20 frame; (C) full system component: foam manifold; and (D) full system component: air manifold.

FIG. 7 shows Average serum antibody titer detected by ELISA testing

### DETAILED DESCRIPTION OF THE INVENTION

The present invention relates to foam vaccination of avians and the preparation of the foam vaccine. The present invention is based on inventors' discovery of a novel foam vaccination system for delivering vaccines to chicks through edible foams. The foams may be used as a vaccine vehicle to improve the uniformity of vaccination while maintaining the level of protection against an infectious pathogen.

The inventors have developed and validated a novel foam vaccination system and method for foam vaccination of avians that outperforms spray vaccination as determined by antibody titers and efficacious protection. Infectious bronchitis vaccine (IBV) was used to model the foam vaccination system and method because it is commonly applied using spray vaccination and is an important poultry disease. Other commercially available vaccines currently in use in a spray or gel vaccination system may also be applied using the foam vaccination system and method according to the present invention. The inventors used broiler chicks/chickens as the model organism due to their high economic impact and pervasiveness. The foam vaccination system may also be used for vaccination of other avian species due to their similar immune system and rearing systems. The inventors have developed a nozzle and foaming agent to produce foam to provide desirable foam qualities such as expansion rate, bubble size, liquid drainage, and deterioration rate, and to ensure that the foaming process and the foaming agent have no significant negative influence the efficacy of the foam vaccine. The inventors have determined the uniformity of the foam application as compared to that of the spray application, and showed that foam-based vaccination successfully provided chicks with a robust immune response.

The inventors have successfully scaled up a foam generating system to a size suitable for vaccination purposes, and developed a foaming agent made from Versawhip, xanthan gum, and deionized (DI) H₂O, for generating repeatedly uniform foams as a vaccine delivery media. The inventors have designed nozzles for producing foams with foaming agents with an expansion rate of about 25.69-33.58, a bubble size of about 3.05-3.52 mm, a liquid drainage of about 55-91% at 10 minutes, and a deterioration rate of about 97.3-98.2 % at 5 minutes. The characteristics of the foams may be controlled by adjusting the components of the foams, for example, ingredients or ratio of gas to liquid.

The inventors have examined the effect of the foaming vaccination system and foaming agent on a vaccine's efficacy. After a mixture of a foaming agent and a vaccine was left to sit for 30, 60, or 120 minutes, the titer was 10^{4.93}, 10^{4.15} and 10^{4.54}, respectively, while the titer of a control was 10^{5.33}. No significant loss in titer (EID₅₀, ≥2 log reduction) was observed when compared to the control. Additionally, after the mixture was foamed, the titer of the foam with the vaccine was 10^{5.53} while its control was 10^{5.31}. Thus, no significant loss of titer was observed when the vaccine was mixed with the foaming agent, whether or not in the form of a foam.

The inventors have investigated the coverage of chicks by a foam application as compared to a spray application. The foam vaccine showed a significantly greater presence at the eyes, choanal cleft, and down of the chicks when compared to a spray application, and was ingested at twice the rate.

Using infectious bronchitis virus (IBV) as a disease model, the inventors have carried out a vaccination trial using foam vaccination and eyedrop and spray vaccination to compare their application uniformity and protection effects. Day-old chicks received the same dose of IBV vaccine through eyedrop, spray, or foams. There were two foam treatments with one group of chicks vaccinated with one pass of full dose vaccine and the other group with two passes of half dose vaccine. Spray and eyedrop vaccination with a full dose of vaccine were carried out according to the manufacturer's instructions. The chicks were raised for 26 days. Nasopharyngeal swabs were performed on day 6 for RT-PCR analysis. Blood samples were collected on day 21 for ELISA tests. Tracheal ring samples and tracheal swabs were collected day 26 for histopathological and VI analysis. RT-PCR showed that eyedrop vaccination and 2-pass foam application had 100% coverage, 1-pass foam had 92.3% coverage, and spray had 72% coverage. The ELISA ranked from highest to lowest titer was 2-pass foam, eyedrop, 1-pass foam, and spray. Tracheal swabs and histological evaluation also support foam vaccination as a potential alternative to spray vaccination. The results of the study demonstrate that foam vaccination provided IBV vaccine uniformly to birds at a protective level similar to that as spray vaccination. Using foam vaccination in the avian industry according to the present invention would ensure that flocks are well protected against infectious diseases when raised on a farm.

The term “foam” as used herein refers to a mass of small bubbles formed by a liquid and a gas. The bubbles may be formed by mixing the liquid and the gas by agitation. The bubbles may have a diameter of about 0.1-10 mm. A stable foam may maintain its structure without breaking down the bubbles into separate liquid and gas phases for a predetermined period of time under predetermined conditions. The stability of a foam may be characterized by its liquid drainage rate (LD) or deterioration rate (DR) upon contact with an absorbent surface. A stable foam may have a LD less than or equal to 95% after 10 minutes or a DR less than 98.2% after 5 minutes.

The term “foaming agent” as used herein refers to an agent whose solution is useful for forming a foam with a gas. The foaming agent may comprise one or more ingredients, each of which may be a chemical compound, a biological molecule or a combination thereof.

The term “surfactant” as used herein refers to an agent that decreases the surface tension or interfacial tension between two liquids, a liquid and a gas, or a liquid and a solid. The foaming agent may be a surfactant that decreases the surface tension of a liquid and gas interface.

The term “viscosity agent” as used herein refers to an agent that alters the thickness of a liquid, for example, a solution.

The term “neutralizing immune response against an infectious pathogen” as used herein refers to an immune response (humoral or cellular) that renders an infectious pathogen unable to cause disease or damage in its host.

The term “titer” as used herein refers to a measurement of concentration for antibodies or virus or vaccine.

The term “vaccination rate” as used herein refers to a percentage of avians (e.g., chicks) in contact with a vaccine after the vaccine is administered to the avians.

The term “immunization rate” as used herein refers to a percentage of avians (e.g., chicks) showing a neutralizing immune response against an infectious pathogen after a vaccine against the infectious pathogen is administered to the avians.

The term “expansion rate (ER)” used herein refers to a volume percentage of a foam over the volume of a liquid used to generate the foam. The ER may be calculated using Equation 1:

$$ER = \frac{{V}_{f}}{( {Vl}_{initial} - {Vl}_{0} )} \qquad ( 1 )$$

Where:
V_{f} is the volume of the foam;
Vlᵢₙᵢₜᵢₐₗ is the volume of the liquid before the foam is generated; and
Vl₀ is the volume of the liquid immediately after the foam is generated.

The term “liquid drainage rate (LD)” as used herein refers to a volume percentage of a liquid produced by the breakdown of a foam over the volume of the liquid used to generate the foam. The LD may be calculated using Equation 2:

$$LD = \frac{{Vl}_{t}}{( {Vl}_{initial} - {Vl}_{0} )} \qquad ( 2 )$$

Where:
Vlₜ is the volume of the liquid at a specific time (t) after the foam is generated, for example, t may be 2-10 minutes;
Vlᵢₙᵢₜᵢₐₗ is the volume of the liquid before the foam is generated; and
Vl₀ is the volume of the liquid immediately after the foam is generated.

The term “bubble size” as used herein refers to an average of diameter of the bubbles in a foam.

The term “deterioration rate” as used herein refers to a weight percentage of a liquid produced by the breakdown of a foam over the weight of the foam before the breakdown. The DR may be calculated using Equation (3)

$$DR = \frac{{CBW}_{final} - {CBW}_{initial}}{foam added} \qquad ( 3 )$$

Where:
CBW_{final} is the final weight of cotton ball;
CBWᵢₙᵢₜᵢₐₗ is the initial weight of cotton balls;
Foam added is the weight of the foam initially added to the cotton balls.

The present invention provides a method for vaccinating one or more avians in need thereof. The vaccination method comprises administering to the one or more avians a stable foam in an amount effective for inducing a neutralizing immune response against an infectious pathogen in the one or more avians. The foam comprises a gas and a liquid. The liquid comprises a vaccine and a foaming agent.

According to the vaccination method, the avian may be a chicken, duck, turkey, pheasant, quail, or goose. The avian may be a chick, pullet, hen, or rooster. The avian may be a day-old chick.

The vaccination method may provide mass vaccinations of the avians (e.g., chicks). The foam may be administered to about 1-1,000, 1-500, 1-100, 1-50, 1-40, 1-30, 1-20 or 1-10 avians (e.g., chick) at once. The administration of the foam to the avians may be repeated one or more times.

According to the vaccination method, the foam may be ingestible. The vaccination method may further comprise ingesting the foam by the one or more avians.

The vaccination method may further comprise contacting the foam with an eye of the one or more avians, and the vaccine enters the one or more avians via the eye.

Upon administration of the foam to an avian, the vaccine in the foam may be in contact with the avian at, for example, an eye, choanal cleft or down. An avian in contact with the vaccine is deemed vaccinated, and the vaccine is deemed to enter the avian, for example, by ingestion or via a bodily fluid, for example, in the eyes.

A vaccinated avian showing a neutralizing immune response against an infectious pathogen is deemed immunized. The infectious pathogen may cause an infectious disease selected from the group consisting of Marek's disease, coccidiosis, mycoplasma gallisepticum infections, infectious bursal disease, encephalomyelitis, fowlpox, laryngotracheitis, Newcastle disease, and infectious bronchitis.

The vaccination method may provide a vaccination rate of at least about 80%, 90%, 95%, 99% or 100%, or about 80-100%, 80-99%, 80-95%, 80-90%, 90-100%, 90-99%, 90-95%,95-100% or 95-99%.

The vaccination method may provide an immunization rate of at least about 80%, 90%, 95%, 99% or 100%, or about 80-100%, 80-99%, 80-95%, 80-90%, 90-100%, 90-99%, 90-95%, 95-100% or 95-99%.

According to the vaccination method, the foaming agent may comprise a surfactant. The surfactant may be present in the foam at a concentration of about 0.1-10, 0.1-5, 0.1-1, 0.1-0.5-10, 0.5-5, 0.5-1, 1-10, 1-5 or 5-10 wt %. The surfactant may comprise sodium stearoyl lactylate (SSL), alkyl glycoside, carrageenan, cholesterol, lanolin, lecithin, monoglyceride, phytosterol, tea saponin extract, one or more proteins, or a combination thereof. The one or more proteins may comprise of an albumin, mucoprotein, globulin, gelatin, casein, milk protein, chickpea protein, gluten, or a combination thereof. The foaming agent may comprise egg white. For example, the foam may comprise a soy protein hydrolysate at, for example, 0.1-10, 0.1-2, 0.5-1 or 3-5 wt %.

According to the vaccination method, the foaming agent may comprise a viscosity agent. The viscosity agent may comprise one or more monosaccharides (e.g., glucose and fructose), disaccharides (e.g., sucrose), polysaccharides (e.g., xanthan gum), or a combination thereof. The foam may comprise xanthan gum at, for example, 0.1-2 or 0.1-0.75 wt %.

According to the vaccination method, the foaming agent may comprise an aqueous solvent. The aqueous solvent may water.

According to the vaccination method, the gas may be air, nitrogen, nitrous oxide, oxygen, carbon dioxide, or a combination thereof.

According to the vaccination method, the foam may have an expansion rate of about 10-50, 20 to 40 or 25-35; a bubble size of about 1-10, 1-5, 2-10, 2-5, 3-10, 3-5, 3-4, 4-10 or 4-5 mm, a liquid drainage of less than about 80%, 85%, 90%, 95% or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at about 5, 10, 15, 1-60, 1-50, 1-40, 1-30, 1-20, 1-10, 5-60, 5-50, 5-40, 5-30, 5-20, 5-15, or 5-10 minutes; and/or a deterioration rate of less than about 90%, 95%, 96%, 97%, 98%, 98.5%, or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at about 1, 2, 3, 4, 5, 6, 7, 8, 9, 10, 1-60, 1-50, 1-40, 1-30, 1-20, 1-10, 1-5, 5-60, 5-50, 5-40, 5-30, 5-20, 5-15, or 5-10 minutes. In one embodiment, the foam has a liquid drainage of less than about 80%, 85%, 90%, 95% or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at 10 minutes. In another embodiment, the foam has a deterioration rate of less than about 90%, 95%, 96%, 97%, 98%, 98.5%, or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at about 5 minutes.

According to the vaccination method, the foam may have the liquid and the gas at a ratio from about 1:10 to about 1:100, from about 1:20 to about 1:200, or from about 1:50 to about 1:100.

The present invention also provides a method for preparing a stable vaccine foam. The preparation method comprises mixing a foaming agent with a vaccine to form a liquid. The vaccine is capable of inducing a neutralizing immune response against an infectious pathogen in an avian in need thereof. The preparation method further comprises mixing the liquid with a gas to form a foam liquid gas mixture; and forcing the liquid gas mixture through a filter to form a stable foam.

According to the preparation method, the foaming agent may comprise a surfactant. The surfactant may be present in the foam at a concentration of about 0.1-10, 0.1-5, 0.1-1, 0.1-0.5, 0.5-10, 0.5-5, 0.5-1, 1-10, 1-5 or 5-10 wt %. The surfactant may comprise sodium stearoyl lactylate (SSL), alkyl glycoside, carrageenan, cholesterol, lanolin, lecithin, monoglyceride, phytosterol, tea saponin extract, one or more proteins, or a combination thereof. The one or more proteins may comprise of an albumin, mucoprotein, globulin, gelatin, casein, milk protein, chickpea protein, gluten, or a combination thereof. The foaming agent may comprise egg white. For example, the foam ay comprise a soy protein hydrolysate at, for example, 0.1-10, 0.1-2, 0.5-1 or 3-5 wt %.

According to the preparation method, the foaming agent may comprise a viscosity agent. The viscosity agent may comprise one or more monosaccharides (e.g., glucose and fructose), disaccharides (e.g., sucrose), polysaccharides (e.g., xanthan gum), or a combination thereof. The foam may comprise xanthan gum at, for example, 0.1-2 or 0.1-0.75 wt %.

According to the preparation method, the foaming agent may comprise an aqueous solvent. The aqueous solvent may water.

According to the preparation method, the gas may be air, nitrogen, nitrous oxide, oxygen, carbon dioxide, or a combination thereof.

According to the preparation method, the foam may have an expansion rate of about 10-50, 20 to 40 or 25-35; a bubble size of about 1-10, 1-5, 2-10, 2-5, 3-10, 3-5, 3-4, 4-10 or 4-5 mm, a liquid drainage of less than about 80%, 85%, 90%, 95% or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at about 5, 10, 15, 1-60, 1-50, 1-40, 1-30, 1-20, 1-10, 5-60, 5-50, 5-40, 5-30, 5-20, 5-15, or 5-10 minutes; and/or a deterioration rate of less than about 90%, 95%, 96%, 97%, 98%, 98.5%, or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at about 1, 2, 3, 4, 5, 6, 7, 8, 9, 10, 1-60, 1-50, 1-40, 1-30, 1-20, 1-10, 1-5, 5-60, 5-50, 5-40, 5-30, 5-20, 5-15, or 5-10 minutes. In one embodiment, the foam has a liquid drainage of less than about 80%, 85%, 90%, 95% or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at 10 minutes. In another embodiment, the foam has a deterioration rate of less than about 90%, 95%, 96%, 97%, 98%, 98.5%, or 99%, about 10-99%, 20-99%, 30-99%, 40-99%, 50-99%, 60-99%, 70-99%, 80-99%, 90-99%, 95-99%, 10-95%, 20-95%, 30-95%, 40-95%, 50-95%, 60-95%, 70-95%, 80-95% or 90-95%, at about 5 minutes.

According to the preparation method, the foam may have the liquid and the gas at a ratio from about 1:10 to about 1:100, from about 1:20 to about 1:200, or from about 1:50 to about 1:100.

The foam prepared according to the preparation method may be ingestible.

According to the vaccination method, the avian may be a chicken, duck, turkey, pheasant, quail, or goose. The avian may be a chick, pullet, hen, or rooster. The avian may be a day-old chick. The infectious pathogen may cause a disease selected from the group consisting of Marek's disease, coccidiosis, mycoplasma gallisepticum infections, infectious bursal disease, encephalomyelitis, fowlpox, laryngotracheitis, Newcastle disease, and infectious bronchitis.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Nozzle and Foam Qualities

The foam generating nozzle was developed through three phases: a concept phase, a prototype phase, and a development phase. The concept phase was used to prove the concept while the prototype phase was designed to test and refine the foam formulation and characteristics. The development phase was finalized for full production. The terms “nozzle,” “development nozzle” and “final nozzle” are used herein interchangeably.

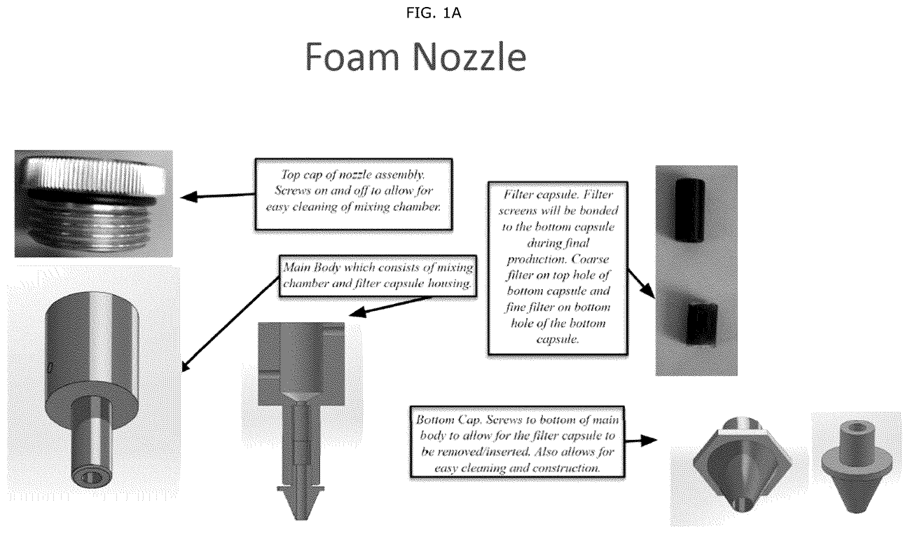

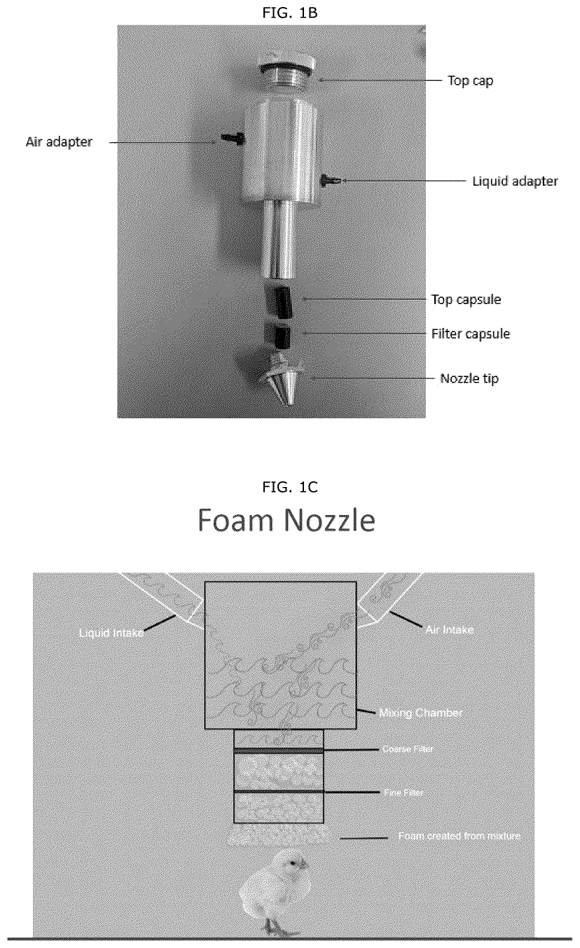

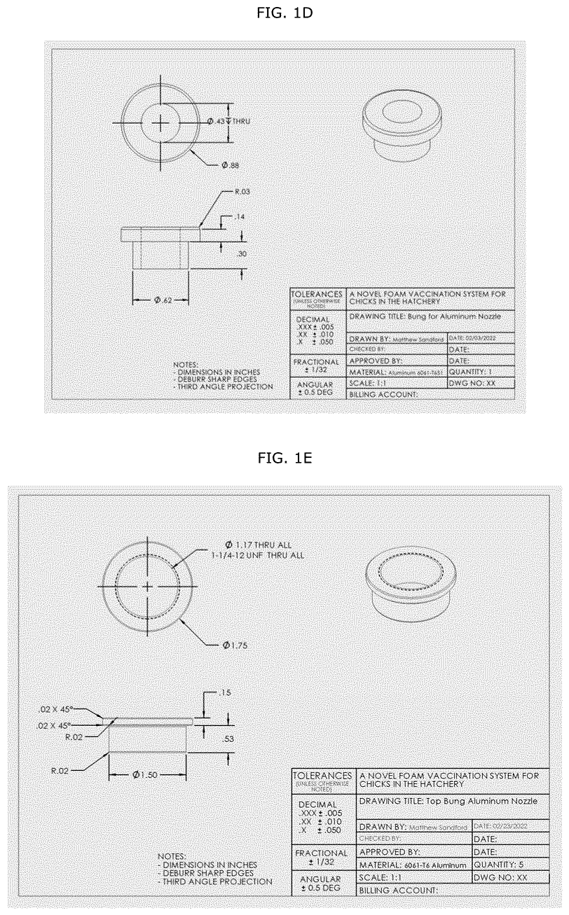

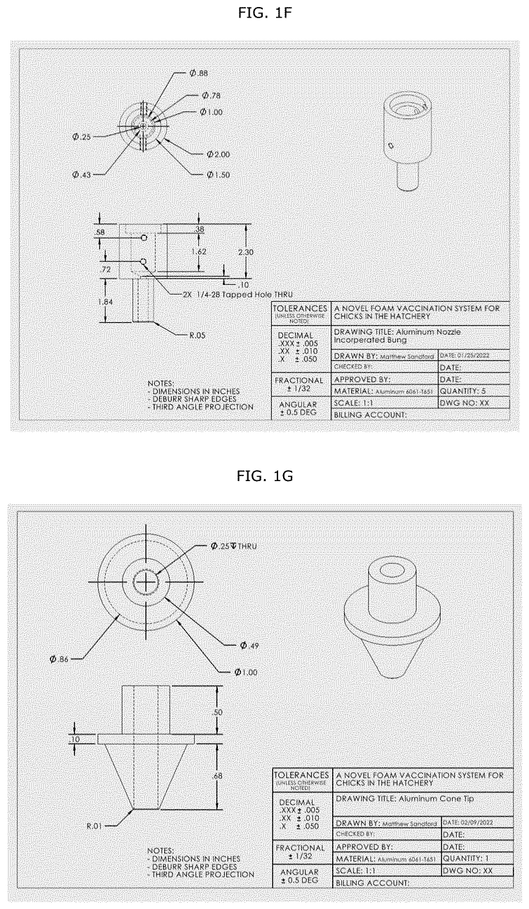

The nozzle was designed using Computer-aided design (CAD) and had its major components machined out of aluminum. The nozzle and its components are outlined in FIGS. 1A-G. The foam nozzle may include a top cap with a screw, a main body having a mixing chamber and a filter capsule housing, a filter capsule with a filter screen, a coarse filter, and a fine filter, and a bottom cap with a screw (FIGS. 1A-B). The foam nozzle may include a top cap, an air adapter, a liquid adapter, a top capsule, a filter capsule and a nozzle tip (FIG. 1B). The nozzle tip may be designed to change distribution of the resulting foam. A filter capsule may have a channel or filter altered to achieve an optimal or desirable foam quality.

FIG. 1C illustrates foam nozzle breakdown. Liquid intake is an injection point of a liquid into a nozzle mixing area. Air intake is an injection point of air flowing into the nozzle mixing area. A mixing chamber is an integral part of the nozzle, in which the air and liquid are mixed by the turbulence caused by the air and liquid injection, and is essential for the beginning of foam production. The air/liquid mixture may be passed through a coarse filter having large pores to create a “coarse foam”. The pores in the filter serve as nucleation sites where the bubbles would form a 3D structure of the foams. After the course foam is generated, the foam may pass through a fine filter, whose pores are smaller than those of the coarse filter. The fine filter serves a two-fold purpose: 1) larger bubbles are split into smaller bubbles, which are less susceptible to breaking or “popping” upon exit of the nozzle, and 2) any air/liquid mix not turned into bubbles has another opportunity to form bubbles and assist in building the foam structure.

The major components of a nozzle include a body (FIG. 1F) and a tip (FIG. 1G). A steel bung may be placed into a top portion of the nozzle for a secure seal. An aluminum bung may be also inserted into a bottom opening of the nozzle so that an aluminum tip is screwed into the nozzle. The internal filter capsule pieces may be 3D printed and 600-micron and 400-micron nylon screens may be attached to the lower capsule with super glue. Two nylon thread adaptors for ¼″ ID tubing may be used for air and liquid input. The top cap (FIG. 1D) and nozzle top (FIG. 1E) were threaded to allow for a tighter seal and easier cleaning. Pneumatic tape was used to seal the nozzle from air leaks.

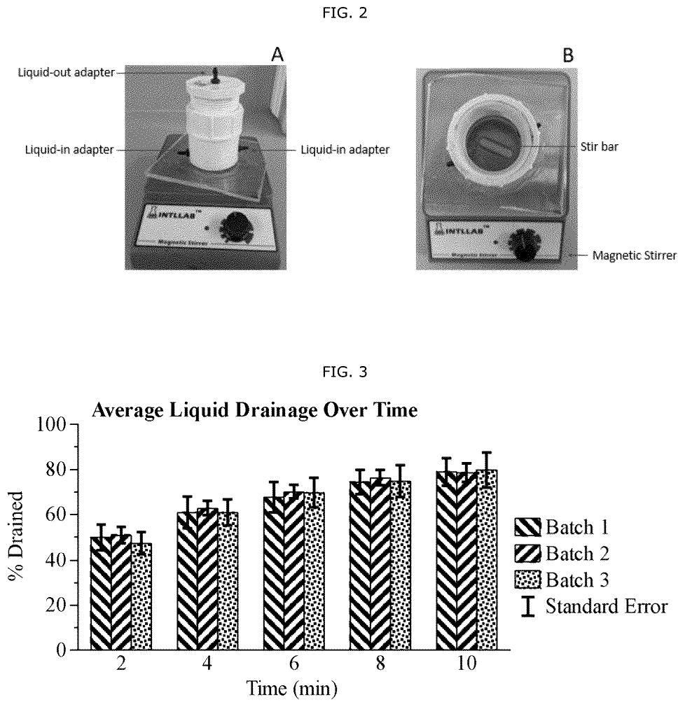

A pump system used for development nozzle testing included two liquid pumps with an addition of a free-standing mixing chamber created from a magnetic stirrer MS-500 using the longest provided stir bar (INTLLAB, Malaysia) (FIG. 2). Two pumps were used to inject liquids into the system: one pump (E20VXCA1S6G1, Stenner, FL) for injecting a vaccine solution and another pump (LB64SA-PTC1-G19, Pulsafeeder, NY) for injecting a foaming agent.

The mixing chamber may have a liquid-out adapter, two liquid-in adapters, a stir bar and a magnetic stirrer (FIG. 2). There may be two liquid-in adaptors as inputs, including one for a reconstituted vaccine by dissolving a dried or lyophilized vaccine into water, and the other one for a foaming agent mixture. Either liquid-in adapter may be used for either liquid. The liquid-out adaptor may be where a mixture of the foaming agent and reconstituted vaccine is forced out and into the manifold and nozzles. The magnetic stirrer and stir-bar may be used to mix the liquids. A medium speed may be used to avoid foaming the mixture within the mixing chamber while still providing equal distribution of the vaccine throughout the foaming agent before it reaches the nozzles.

The filter dimensions (e.g., length, width, thickness, or pore size), nozzle dimensions (e.g., length, width, thickness, or channel dimensions or shape), body dimensions (e.g., length, width, thickness, channel dimensions or shape, or number of liquid input channels), filter materials, nozzle materials, and/or body materials may be altered to produce a foam with desirable characteristics, for example, expansion rate, liquid drainage, bubble size, and deterioration rate). Inclusion of a vaccine in a foam does not change the characteristics of the foam.

### 1. Expansion Rate

An expansion rate (ER) of a foam is a volume percentage of the foam over the volume of a liquid used to generate the foam. The ER may be calculated using Equation 1:

$$ER = \frac{{V}_{f}}{( {Vl}_{initial} - {Vl}_{0} )} \qquad ( 1 )$$

Where:
V_{f} is the volume of the foam;
Vlᵢₙᵢₜᵢₐₗ is the volume of the liquid before the foam is generated; and
Vl₀ is the volume of the liquid immediately after the foam is generated.

The expansion rate of a foam may be tested by placing a foam nozzle on top of an acrylic cylinder, for example, 51 mm in diameter and 152 mm in height, and dispensing a foam until the top layer of the foam is just below the nozzle tip. The initial amount of the liquid may be measured into graduated cylinders that serve as a reservoir for the foaming agent and/or a vaccine simulant. After the foam is dispensed, the volume of the liquid in the graduated cylinder(s) may be measured and a ruler may be used to measure the height of the foam from the bottom of the foam to the top of the foam. In case that there is a slanted top layer, the midpoint of the slant may be used. The height of the foam may be used to calculate the total volume of the foam produced. For a final nozzle, the expansion rate may be tested on three separate batches of a foaming agent, and five times for each batch. A batch is a fresh mixture of the foaming agent components.

### 2. Liquid Drainage

A liquid drainage rate (LD) of a foam is a volume percentage of a liquid produced by the breakdown of a foam over the volume of the liquid used to generate the foam. The LD may be calculated using Equation 2:

$$LD = \frac{{Vl}_{t}}{( {Vl}_{initial} - {Vl}_{0} )} \qquad ( 2 )$$

Where:
Vlₜ is the volume of the liquid at a specific time (t) after the foam is generated, for example, t may be 2-10 minutes;
Vlᵢₙᵢₜᵢₐₗ is the volume of the liquid before the foam is generated; and
Vl₀ is the volume of the liquid immediately after the foam is generated.

The liquid drainage may be performed by placing a nozzle on top of an acrylic cylinder, for example, 51 mm in diameter and 152 mm in height, with a ¼″ hole drilled into the bottom of the cylinder. This orifice allows the liquid produced by the breakdown of the foam to flow out of the container but not the foam. Next, a graduated cylinder may be placed underneath the acrylic cylinder to catch the liquid running out of the acrylic cylinder. After measuring the initial amount of the liquid, the foam may be dispensed until the top layer of foam is just below the nozzle tip. The amount of the liquid used for generating the foam may be recorded at this time. At every two minutes for 10 minutes, the amount of the liquid present in the graduated cylinder may be recorded. Three independent batches of the foaming agent may be tested five times for each batch.

### 3. Bubble Size

The bubble size of a foam is an average of diameter of the bubbles in a foam. The bubble size may be determined by photographing the foam and then measuring the diameter of the individual bubbles using a photo editing software, for example, GNU Image Manipulation Program 2 (GIMP2). For bubble size tests done with a final nozzle, a foam may be dispensed onto a flat sheet of 1/16″ Lexan. A ruler may be placed next to the foam as a scale. A picture may then be taken from underneath the Lexan. These pictures may then be loaded into GIMP2 and processed by drawing a 1″×1″ square in the center of the foam pile. Every bubble having its longest diameter within the box may be measured. Each test may be repeated five times and the average may be calculated.

### 4. Deterioration Rate

The deterioration rate (DR) of a foam is a weight percentage of a liquid produced by the breakdown of a foam over the weight of the foam before the breakdown. The DR reflects the stability of the foam, for example, on an absorbent surface such as a chick body. The DR may be calculated using Equation (3)

$$DR = \frac{{CBW}_{final} - {CBW}_{initial}}{foam added} \qquad ( 3 )$$

Where:
CBW_{final} is the final weight of cotton ball;
CBWᵢₙᵢₜᵢₐₗ is the initial weight of cotton balls;
Foam added is the weight of the foam initially added to the cotton balls.

Deterioration rate may be tested by constructing a tinfoil holder 4″×4″ with 1″ high walls. Nine cotton balls may be weighed and then placed in a one layer, 3×3 grid in the tinfoil holder. The tinfoil holder containing the cotton balls may be then placed on a scale, which may then be zeroed. A foam may then be applied to the cotton balls and the weight of the added foam may be recorded. The weight of the cotton balls may be monitored to observe any evaporative loss for 5 or 10 minutes before the remaining foam may be scraped off and the cotton balls may be weighed. This process may be repeated five times for each of the 5- and 10-minute testing.

### 5. Results and Conclusion

Nine (9) foaming agent formulations were generated using the forming agent ingredients and tested using a final nozzle under the conditions as described in Table 1.

TABLE 1
Foaming agent formulations and test conditions

| | Foaming agent | |
|---|---|---|
| Formulation | ingredients (wt %) | Test conditions |
| 4 | 100% egg white | room temperature |
| 5 | 0.5% SSL, 74.6% egg white, | heated to 54 C. |
| | 24.9% DI H2O | |
| 6 | 1% SSL, 74.3% egg white, | heated to 54 C. |
| | 24.7% DI H2O | |
| 1 | 75% egg white, 25% DI H₂O | room temperature |
| | | at 21 C. |
| 1.1 | 75% egg white, 25% DI H₂O | heated to 72 C. |
| 1.2 | 75% egg white, 25% DI H₂O | heated to 62 C. |
| 7 | 4.8% versawhip, 0.2% xanthan | |
| | gum, 95% DI H₂O | |
| 7.1 | 4.6% versawhip, 0.4% xanthan | |
| | gum, 95% DI H₂O | |
| 7.2 | 4.4% versawhip, 0.6% xanthan | |
| | gum, 95% DI H₂O | |

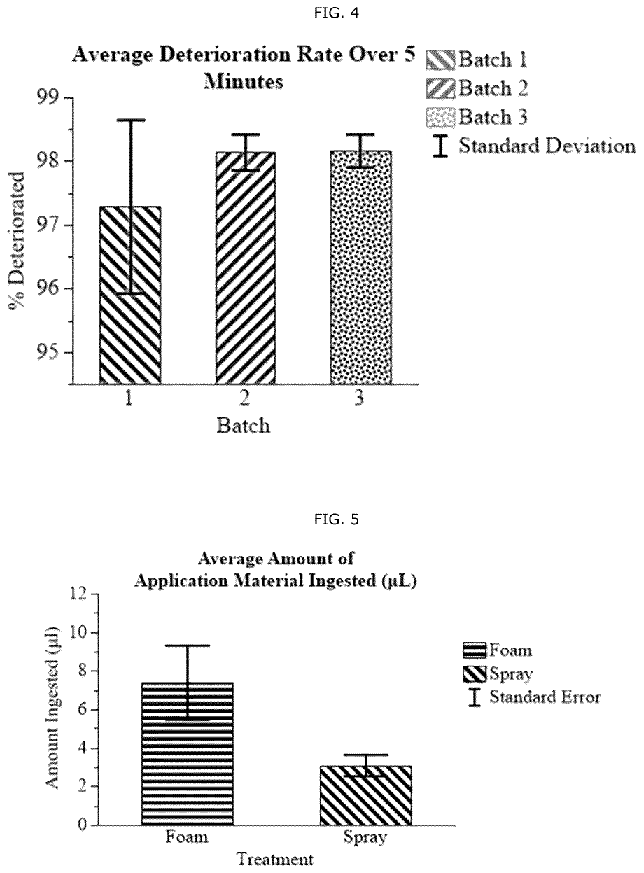

Foaming agent formulation 7.1 showed an average expansion rate of about 25.69-33.58, an average bubble size of about 3.05-3.52 mm (Table 2), an average liquid drainage of about 59-91% at 10 minutes (FIG. 3), and an average deterioration rate of about 97.3-98.2% at 5 minutes (FIG. 4).

TABLE 2
Average expansion rate and bubble size

| Batch # | Expansion Rate | Bubble Size (mm) |
|---|---|---|
| 1 | 25.69 ± 5.33 | 3.30 ± 2.19 |
| 2 | 27.53 ± 3.52 | 3.05 ± 1.65 |
| 3 | 33.58 ± 5.35 | 3.52 ± 1.58 |

The results of these experiments show that the final nozzle can produce stable foams.

### Example 2. Foam Vaccine Efficacy

### Materials and Methods

### Vaccine

The vaccine used in this study was Merck MILDVAC-MA5 (Kenilworth, NJ) a live-attenuated vaccine. The product contains instructions for coarse spray application, and drinking water application. Each vial of vaccine contained 10,000 doses. Vaccines were titrated alongside treatments to serve as a positive control. Titrations were carried out by diluting a fresh vaccine vial in 100 mL of DI H₂O. This then was diluted again with DI H₂O to match the dilution that would occur with the foaming agents. These dilutions are described below.

The resulting mixture was then mixed at a ratio of 1:2 with Tryptose phosphate broth (TPB) containing Penicillin, Streptomycin, and amphotericin B. 1:10 serial dilutions were then carried out to the same level as the treatments, dependent on the experiment. The chosen dilutions then had 0.2 ml inoculated via the chorioallantoic sac route (CAS) route into 5 10-day-old embryonated specific pathogen free eggs. The eggs were candled for seven days and placed in a 4° C. refrigerator on the last day. Any eggs that had died on day 1 or 2 were removed from analysis. The remaining eggs were dissected and examined for IBV specific lesions including stunting, clubbed down, and hemorrhage. The Reed-Muench method was then used to calculate the titer of an embryo infectious dose (EID₅₀).

### Experimental Design

The foaming agent formulation 7.1 was generated as described in Example 1. The vaccine was diluted in 100 mL of DI water. Then 3 mL of the vaccine was mixed with 24 mL of the foaming agent formulation 7.1 to simulate the amount of vaccine used during foam dispensing. These mixtures were allowed to sit for 30, 60, or 120 minutes before being mixed at a 1:2 ratio with TPB containing the same antibiotics. This was allowed to rest for 30 minutes at room temperature. From this solution, a 1:10 serial dilution took place from 10⁻¹ to 10⁻⁶. A volume of 0.2 mL of serial passages from 10⁻³ to 10⁻⁶ were then inoculated in 10-day old specified pathogen-free (SPF) eggs via the CAS route. This process was repeated with DI water being mixed at the same ratios as the foaming agent formulation with the vaccine and being run through the foaming machine at each of the prescribed levels to generate a vaccine foam. Timepoints, dilutions, and inoculations were the same. A positive control was created by using the 10⁰ vaccine/DI water solution that was diluted similarly with TPB containing Penicillin, Streptomycin, and Ampohtercin B, and then serially diluted from 10⁻¹ to 10⁻⁵ and inoculated into 5 10-day-old embryonated SPF eggs per dilution. Over the course of a week (i.e., 7 days) the eggs were candled daily and any that died after day 2 were counted as IBV specific and retained in a 4° C. refrigerator. At the end of the week, any eggs remaining were moved to the 4° C. refrigerator and held for a period of 48 hours. After 48 hours, the eggs were opened and examined for the previously mentioned IBV-specific lesions. The Reed-Muench method was used to calculate titer EID₅₀.

### Statistical Analysis

Titers were calculated using the Reed-Muench method to give an EID₅₀. Interpretation of results was based on “Note for guidance on virus validation studies: The design, contribution and interpretation of studies validating the inactivation and removal of viruses” where reductions log₁₀=no significant reduction, 1-2 log₁₀=mild reduction, 2-4 log₁₀=moderate reduction, and >4 log₁₀=severe reduction (Note for Guidance on Virus Validation Studies: The Design, Contribution and Interpretation of Studies Validating the Inactivation and Removal of Viruses, 1996; Ruppach, 2014).

### Results and Conclusions

Table 3 reflects the titer change of the foam vaccine generated after the vaccine and the foaming agent formulation 7.1 were mixed for at 30, 60 or 120 minutes. There was no significant reduction (≥log₁₀) between the titer at 30 minutes and that at 120 minutes. At 60 minutes, there was a slight, but inconclusive loss (1.18 log₁₀) of titer.

TABLE 3
Results of vaccine efficacy trial for foaming agent formulation 7.1

| Treatment | Vaccine Vehicle | Titer (EID₅₀) |
|---|---|---|
| Positive control | DI H2O | 10^{5.33} |
| 30 minutes | Foaming agent | 10^{4.93} |
| 60 minutes | Foaming agent | 10^{4.15} |
| 120 minutes | Foaming agent | 10^{4.54} |

This experiment shows that the foaming agent has no significant effects on the viability of vaccines.

### Example 3. Uniformity of Foam Application

### Ingestion and Coverage Testing Explanation

For measuring ingestion and coverage, a 1% fluorescein solution was made using fluorescein and the vaccine simulant (H₂O). After being mixed in the foaming system, a further 1:8 dilution of the fluorescein solution was prepared. For measuring ingestion and coverage of a spray apparatus, a 1% fluorescein solution was made using fluorescein and DI H₂O. This was further diluted to match the dilution that occurred for the foam liquid after being mixed by the machine. Ingestion analysis samples were processed by measuring the amount of fluorescein solution in the gastrointestinal tract of chicks. The amount ingested was calculated using a standard curve for both foam and spray applications.

For the coverage analysis, each chick carcass was scored under blue lightbulbs in a dark room allowing for the fluorescein to be visible on the chicks' bodies. A binary scoring system with two scores was used to determine the coverage to vaccine solution scored as the appearance of fluorescein at different parts of chick body. A general coverage score was determined by summing the score for the presence of fluorescein (score of 1) or absence of fluorescein (score of 0) for each individual chick on any one of six body parts, including down, eyes, nares, beak, choanal cleft, and tongue, while an inoculation score was based on the presence of fluorescein on any one of three body parts, including eyes, nares, and choanal cleft, that are responsible for inoculation.

### Results and Conclusion

Mass application via a foam application reaches the eyes (p<0.001) and choanal clefts (p<0.001) of the chicks more effectively than that via a spray application. After the foam application, eighty-five percent of the chicks had signs of fluorescein entering the eye, while 55% showed fluorescein in the choanal cleft. This is compared to the chicks that underwent the spray vaccination, where only 47.5% of the chicks showed fluorescein entering the eye and only 35% showed fluorescein in the choanal cleft. Additionally, only 72% of the chicks showed fluorescein on their down after the spray application as opposed to a 95% coverage seen on the chicks after the foam application (Table 4).

TABLE 4
Percentage of chicks covered with application
material following a foam or spray application

| | | | | | | % Choanal |
|---|---|---|---|---|---|---|
| | % Eyes | % Nares | % Beak | % Down | % Tongue | Cleft |
| Foam | 85.0^{a} | 60.0 | 75.0 | 95.0^{a} | 62.5 | 55.0^{a} |
| Spray | 47.5^{b} | 75.0 | 67.5 | 72.5^{b} | 60.0 | 35.0^{b} |

Eyes p < 0.001; choanal cleft p < 0.001; down p < 0.01.
n = 40 for both treatments.

The chicks undergoing the foam application ingested 7.4 μL of the application material, which was significantly greater (p<0.05) than the 3.1 μL of the application material ingested by the chicks after the spray application (FIG. 5).

This data is in line with reports that showed lower overage rate of spray vaccination. Overall, this study shows that foam application reaches chicks and the inoculation target areas of the chicks more effectively than spray application. The average coverage rate of a vaccination application at different target body parts may be used to indicate the efficiency of the vaccination application.

### Example 4. Comparative Vaccination Test of Chicks: Spray vs Foam

### Experimental Design

### Vaccination Process

Two hundred and ninety straight-run day-old conventional broiler chicks were obtained from a local commercial hatchery within a two-hour driving distance from the research facilities. The chicks were randomly divided into seven (7) groups for different vaccination treatments. After vaccination treatment, each group of chicks was placed into a naturally ventilated small colony house (7.4 ft×7.4 ft). The temperature of each house was set to 95° F. for day-old and was lowered gradually over the course of the experiment until the final day where the average house temperatures were approximately 75° F. The temperature was adjusted by controlling the vent opening, a heat lamp (250 W), and an electric heater (1,500 W) with an internal thermostat. Thermal environmental related behaviors, such as panting, huddling, or spreading along house edges were used to modify the thermal condition. Birds were fed ad libitum with commercial starter and grower feed during the length of the trial. Water was also supplied ad libitum with a double wall poultry drinker. Each house was checked twice per day for temperature, feed, water, and disease; once between 7 am to 10 am and once between 4 pm to 6 pm.

### Vaccine

The vaccine used in this study was a live-attenuated vaccine (MILDVAC-MAS, Merck, Kenilworth, NJ). Each vial of vaccine contained 10,000 doses. The vaccine was titered by mixing one vial from a serial in 100 mL of DI H₂O then mixing at a ratio of 1:2 with sterile tryptose phosphate broth (Molecular Toxicology, NC) containing streptomycin (10,000 IU/ml), penicillin (10,000 IU/ml), and amphotericin B (25 IU/ml) (Lonza BioWhittaker, MD; Fisher BioReagents, MA) and was allowed to incubate at room temperature for 30 minutes. This stock was then serially diluted ten-fold from 10⁻¹ to 10⁻⁷. Then 0.2 mL dilutions from 10⁻³ to 10⁻⁷ were inoculated into five, 10-day old specific pathogen free (SPF) eggs via CAS route each. The eggs were candled for seven days following inoculation and placed in a 4° C. refrigerator on the last day. Any eggs that had perished on day 1 or 2 post-inoculation were removed from analysis. The remaining embryos were then examined for IBV specific lesions which included stunting, clubbing of feathers, presence of “button body”, and hemorrhage. The Reed-Muench method (Reed & Muench, 1938) was then used to calculate the embryo infectious dose, which was 10^{5.33} EID₅₀ per ml.

### Virus Challenge

The challenge virus used in this study was an M41 strain and titered similarly to the vaccine but the serial dilutions were from 10⁻¹-10⁻⁵ and 0.2 mL dilution was inoculated from all five dilutions. The resulting titer was 10^{5.77} EID₅₀ per ml. Virus stocks were diluted at challenge so that all birds in study were challenged with 10⁴ EID₅₀ via eyedrop.

### Treatment and Sampling

Seven (7) groups were used in this study. All birds were wing banded and placed into groups before treatments. Negative control birds and positive control birds were placed in respective houses before any vaccination took place. Group 1 was the negative control group containing 40 birds that received no vaccination and no challenge over the course of the trial. Group 2 was the positive control group containing 20 birds that were not vaccinated and were challenged on day 21 of the study. Group 3 was the vaccine control group that contained 40 birds that received eyedrop vaccination but were not challenged. Group 4 was the foam 1-application group containing 40 birds that received one full dose of foam vaccination and was challenged on day 21 post-vaccination. Group 5 was the eyedrop vaccination group containing 40 birds that were vaccinated via eyedrop and challenged at 21 days post-vaccination. Group 6 was the foam 2-application group containing 40 birds that received two half-doses of foam vaccination (1 full dose total) and were challenged at 21 days post-vaccination. Group 7 vaccination was the spray vaccination group containing 40 birds that were spray vaccinated and challenged at 21 days post vaccination. On day 6 of the trial, nasopharyngeal swabs were collected for qRT-PCR analysis. On day 21 blood samples were collected from half of the birds in each treatment group to determine antibody titer via ELISA testing and were subsequently challenged with M41. On day 26 of the trial, remaining birds were euthanized through cervical dislocation. Tracheal sections were collected for histopathological analysis and tracheal swabs were collected for virus isolations. Table 5 provides a comprehensive schedule for the sampling and vaccination timeline, sample collection and processing.

TABLE 5
Treatment groups and sampling schedule for vaccination trial

| | | | Sampling | Sampling | Challenge | Sampling |
|---|---|---|---|---|---|---|
| Group | n | Vaccinated | Day 6 | Day 21 | Day 21 | Day 26 |
| Negative control | 40 | N | Y | Y | N | Y |
| Positive Control | 20 | N | Y | Y | Y | Y |
| Vaccine control | 40 | Y | Y | Y | N | Y |
| Eyedrop | 40 | Y | Y | Y | Y | Y |
| Foam 1-pass | 40 | Y | Y | Y | Y | Y |
| Foam 2-pass | 40 | Y | Y | Y | Y | Y |
| Spray | 40 | Y | Y | Y | Y | Y |

### Nasopharyngeal Swabs and qRT-PCR

Swabs were collected from birds on the 6^{th} day of the study (i.e., 5 days post vaccination). Fifteen-milliliter centrifuge tubes had 3 mL of Sterile TPB containing antibiotics were prepared and frozen at −20° C. before the sampling. Before sampling, these tubes were placed on ice to thaw as the sampling occurred. Nasopharyngeal swabs were obtained using flocked nylon swabs. Swabs were broken off into the tubes of media after being gently swirled at which point the tubes were sealed and labeled according to bird number. These samples were then immediately placed on ice and transferred to a −20° C. freezer until qRT-PCR evaluation.

To isolate RNA, the frozen swabs were thawed and 250 μL was plated into a 96 well plate for analysis while the rest was refrozen. A 5× viral isolation kit (MagMAX™-96, Thermo Fisher Scientific, MA) following the manufacturer's instructions was used to extract viral RNA with an One-step RT-PCR kit (Ag Path-ID™, Thermo Fisher Scientific, MA) in combination with a Kingfisher FLEX (Thermo Fisher Scientific, MA) was used to quantify the titer using general IBV primers (Primer 1 5′-GCTTTTGAGCCTAGCGTT-3′ (SEQ ID NO: 1); Primer 2 5′-GCCATGTTGTCACTGTCTATTG-3′ (SEQ ID NO: 2) and a general IBV FAM probe, which consists of 6-carboxy-fluorescein probe (FAM), CACCACCAG, the ZEN™ internal quencher (Integrated DAN Technologies, Coralvilles, Iowa, USA), AACCTGTCACCTC (SEQ ID NO: 3) and 3′ Iowa Black® FQ (3IABkFQ) as a second quencher linked in a chain from the 5′ end to the 3′ end. The primers (i.e., Primer 1 and Primer 2) and the general IBV FAM probe) use DNA nucleotides as their main component and target an 143-bp product in the 5′-UTR of the IBV genome. The FAM does not fluoresce unless it is cleaved from the general IBV FAM probe structure by a polymerase used during a RT-PCR process. The strength of the resultant fluorescent signal is used to quantitate viral load. The quenchers (i.e., ZEN™ and 3IABkFQ) absorb the FAM's emission spectra (i.e., fluorescent signal) when the quenchers are kept in dose proximity so that an un-cleaved general IBV FAM probe will not be added to the quantitation. A primer-probe master mix was made by combining 85 μl of nuclease free water with 25 μl of primer 1 (60 μM), 25 μl of primer 2 (60 μM), and 15 μl of probe (30 μM). The 25 μl reaction contained 12.5 μl 2× RT-PCR buffer mix (AgPath-ID™, Thermo Fisher Scientific, MA), 1 μl 25× RT-PCR enzyme mix (Applied Biosystem, MA), 2 ul of primer-probe master mix, 6 μl RNA template, and 3.5 μl of nuclease free water. The reaction was carried out in a 7500 Real Time PCR System (Thermo Fisher Scientific, MA) out at 45° C. for 10 minutes, 95° C. for ten minutes, and 40 cycles of 95° C. for 15 s , and 60° C. for 45 s. A standard curve was created by diluting one 10,000 dose vial of vaccine in 100 mL of DI H₂O and then making 1:10 serial dilutions from 10⁻¹ to 10^{-4 .} The dilutions were then plated in triplicate.

### Blood Samples and ELISA

Blood samples were taken using 3 mL syringes with 20-gauge needles from the wing vein on day 21. Birds were held by one person and the wing vein was exposed while another person collected 1-2 mL of blood. Samples were then placed on ice during sampling and then transferred to a 4° C. refrigerator to facilitate clotting. Unfortunately, the samples were then transferred to a −20° C. freezer before the serum could be collected resulting in hemolysis of the blood samples with some samples yielding no serum. Nonetheless, samples were then thawed, and serum samples were decanted into 1.5 mL centrifuge tubes and refrozen at −20° C.

Samples were thawed before sample analysis. An indirect ELISA kit (IBV, BioChek, UK) was used to perform the ELISA following the manufacturer's instructions. An IDEXX Emax plate reader using xChek software was used to read the plates.

### Tracheal Sections and Histopathology

A 1-3 cm section of trachea was harvested from the first two inches of the trachea and placed into a labeled sampling jar containing 5 mL of 10% neutral buffered formalin (Thermo Fisher Scientific, MA) for fixation. Samples were kept at room temperature until they were processed and examined. Tissues were trimmed and embedded in paraffin at 4 μm thickness and then stained with Hematoxylin and Eosin. Microscopic analysis was blinded. Samples were inspected for lesions that were representative of IBV infection. These lesions were counted and summed to give a score for each sample: samples with scores 0-3=suspected no viral replication, samples with a score of 4=indeterminate, and samples with scores of 5+=suspected viral replication.

### Tracheal Swabs and Virus Isolations

After the tracheal sections were taken, tracheal swabs were collected by inserting a nylon flocked swab into the trachea and gently rotating and moving the swab up and down an area of approximately 4 in. The swabs were then immediately placed in 3 mL of partially frozen, sterile TPB and placed on ice. These samples were then promptly transported to a −80 ° C. freezer upon completion of sample harvesting for each group. Later, 1 mL of these samples were aliquoted into individual mini centrifuge tubes for the virus isolations and frozen at −20 ° C.

Virus isolations took place for groups 2-7 by thawing the samples kept at −20° C. on ice and then centrifuging the samples at 15,200 rpm for six minutes to pellet out bacteria. Eight hundred microliters of this sample was then pipetted into a fresh 1.5 mL snap-cap centrifuge tube. Next, 0.2 mL sample was inoculated into four 10-day old SPF eggs for each sample via the CAS route. Eggs were candled for 7 days. Any eggs that died on days 1-3 were discarded. Eggs then were placed in a cold room for 24 hours before embryos were examined for IBV specific lesions. Any egg that showed signs of bacterial contamination was discarded. An egg was considered contaminated if the chorioallantoic fluid had a green or black coloring. Any sample that had less than 3 eggs remaining was not used in the analysis.

### Vaccination Systems

### Spray Vaccination

Spray vaccination was carried out using a cabinet sprayer, that utilizes air pressure of 50 PSI and two nozzle heads to dispense seven mL of vaccine onto a standard 100-chick tray. One vial of 10,000 doses of the vaccine was reconstituted in DI H₂O to a concentration of 100 doses per seven ml. Forty chicks were constrained to 40% floor area of a standard commercial chick tray using a cardboard divider. The chicks were sprayed and contained within the cabinet for five minutes to allow for the spray to settle. The tray was then removed and left in the open for another five minutes before the chicks were transported to the farm and placed in the houses.

### Foam Vaccination

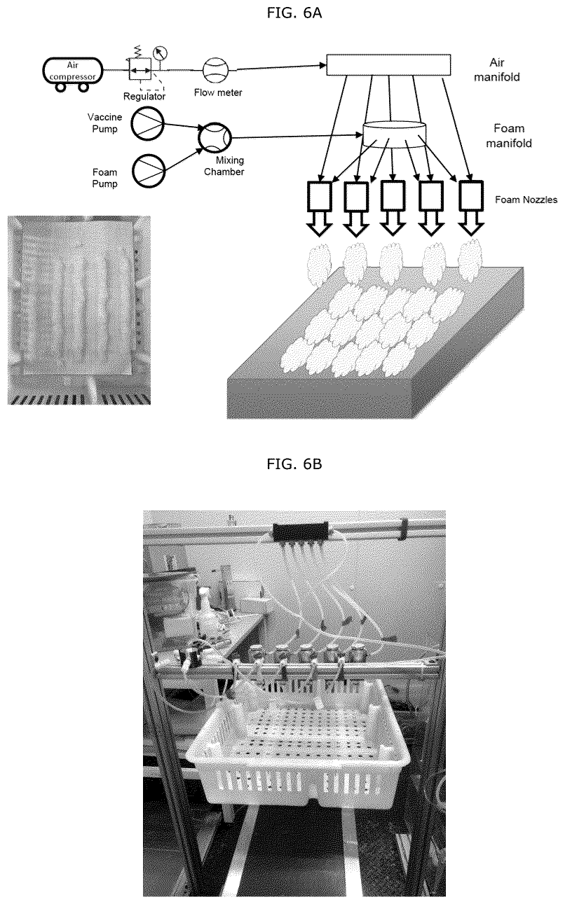

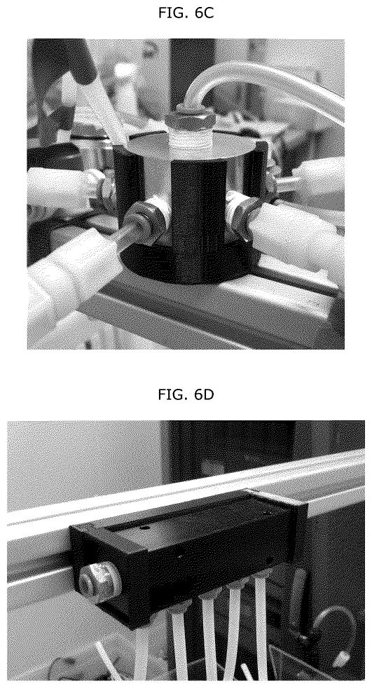

The foam vaccination system (FVS) uses five foam development nozzles (see FIGS. 1A-G for detailed outline of nozzles) to deliver a total of 27 mL of vaccine to a standard 100-chick tray per pass. A basic outline and description of the FVS and its components can be seen in FIGS. 6A-D.

An air compressor may be used as the air supply for the foam nozzles.

An air regulator may be used to keep the applied air pressure in the system at a safe, consistent level.

A flow meter may be used to regulate the volume of air that is flowing through the system. If adjusting the liquid:air ratio, this is typically what is used to regulate the air portion and can affect the speed at which foam is deployed.

An air Manifold may be used to equally distribute the air between the nozzles.

A vaccine pump may be a liquid pump used to pump the vaccine into the mixing chamber. By adjusting the flow rate on the pump, the dosage may be increased or decreased.

A foam pump may be a liquid pump used to pump the foaming agent to the mixing chamber and may be the primary pump responsible for moving liquid through the mixing chamber into the nozzle. By adjusting the flow rate here, the liquid:air ratio on the side of “liquid” may be adjusted to alter foam qualities and speed at which foam is deployed.

A mixing chamber may be used to ensure that the liquid entering the nozzles to be foam has an equal distribution of vaccine before being deployed.

A foam manifold may distribute foaming agent/reconstituted vaccine equally among nozzles.

Foam nozzles are an integral part of the FVS where foam creation and deployment occur.

Filters may be used to adjust foam quality and nozzle tips or number of nozzles may be changed to achieve optimal distribution.

For foam vaccination, there are two pumps: one for vaccine and one for the foaming agent (as described u). 3.5 mL of vaccine is used for every 23.5 mL of foaming agent used for 1 pass of a standard commercial 100-chick tray. These are pumped separately into a mixing chamber (FIG. 2) before being pumped out to the nozzles. This setup ensures that the vaccine is evenly distributed throughout the foam at time of application. For the one-application (i.e., 1-application) vaccination, one 10,000 dose vial was diluted so that 100 doses would be applied over a standard 100-chick tray after one pass. For the two-application (i.e., 2-application) vaccination, the vial was diluted again so that only 50 doses would be applied over the entire tray in one-application.

For the one-application foam vaccination, 40 chicks were restricted to 40% floor area of a standard commercial chick tray. This was then placed on the conveyor belt. The belt and system were turned on at the same time allowing for one-application of the foam to be applied at a speed of 0.1 m/s. The tray was then set aside for 10 minutes to allow for interaction with the foam. After 10-minutes, chicks were transported to the farm and placed.

For the two-application foam vaccination, 40 chicks were restricted to 40% floor area of a standard commercial tray. This was then placed on the conveyor belt. The belt and system were turned on at the same time allowing for one-application of the foam to be applied at a speed of 0.1 m/s. The tray was then brought back to the start of the conveyor belt and a second pass was initiated. The tray was then set aside for 10 minutes. After 10 minutes, the birds were brought to the farm and placed.

The foaming agent/vaccine ratio, number of applications, and speed of the belt may be altered to achieve the same expected results.

### Eyedrop Vaccination

Eyedrop vaccination was carried out using a one mL 26-gauge syringe One 10,000 dose vial was reconstituted to a concentration of one dose per 0.3 ml. Vaccine was applied dropwise to the eyes. Chicks were handled individually for this application.

### Statistical Analysis

Excel and JMP 16 were used to analyze the data. One-way ANOVA and Tukey Post-hoc tests were used to analyze significance. Results for averages are reported as mean±standard error.

### Results and Conclusions

### Vaccine Titers

The vaccine control group (n=39) had an average titer of 22081±2431 (10^{4.34} EID₅₀) which was significantly higher than the spray, foam 1-application, negative control, and positive control groups. The eyedrop vaccination group (n=39) had an average titer of 18751±1862 (10^{4.27} EID₅₀) which was significantly higher than the foam 1-application, negative control, and positive control groups. The foam 2-application vaccination group (n=39) had an average titer of 14942±1969 (10^{4.17} EID₅₀) which was significantly higher than the negative and positive controls. The spray vaccination group (n=36) had an average titer of 11199±3607 (10^{4.05} EID₅₀) while foam 1-application vaccination group (n=39) had an average titer of 891±2306 (10^{2.95} EID₅₀), with neither of these significantly higher than the negative or positive controls. The negative control group (n=40) and positive control group (n=19) had miniscule average titers of 1.4±0.9 (10^{0.14} EID₅₀) and 0.1±0.1 respectively.

The data from the day 6 swabs suggests that foam vaccination achieved similar titers of vaccine spray and eyedrop vaccination, dependent on the amount of foam deployed. The 2-application foam had a similar average titer to both groups receiving eyedrop vaccination and spray vaccination while foam 1-application vaccinations average titer was significantly lower than both eyedrop groups but was similar to spray vaccinations titer.

In the vaccine control group, the eyedrop group, and the two-application foam group, 100% of the samples tested were positive for vaccine. Foam with one-application had 92.3% of samples that were positive while spray vaccination only had 72.2% positive. The negative control had 10% of those tested positive while the positive control had 5.3% positive. The distribution of titer magnitude for each of the groups is outlined in Table 6.

TABLE 6
Distribution of titers based on qRT-PCR testing of day 6 nasopharyngeal swabs

| Treatment | 0 | 0.01-0.99 | 1-1.99 | 2-2.99 | 3-3.99 | 4-4.99 | 5-5.99 |
|---|---|---|---|---|---|---|---|
| Negative Control | 36 | 3 | 1 | 0 | 0 | 0 | 0 |
| Positive Control | 18 | 1 | 0 | 0 | 0 | 0 | 0 |
| Vaccine Control | 0 | 0 | 0 | 0 | 8 | 31 | 0 |
| Foam 1-pass | 3 | 2 | 3 | 6 | 16 | 9 | 0 |
| Eyedrop | 0 | 0 | 1 | 0 | 9 | 29 | 0 |
| Foam 2-pass | 0 | 1 | 4 | 1 | 9 | 24 | 0 |
| Spray | 10 | 1 | 5 | 1 | 3 | 15 | 1 |

### 6. Antibody Assessment

The ELISA tests conducted on the blood samples taken on day 21 of the study suggest that foam vaccination, one- or two-application, elicit similar titers of serum antibodies to both spray and eyedrop vaccination.

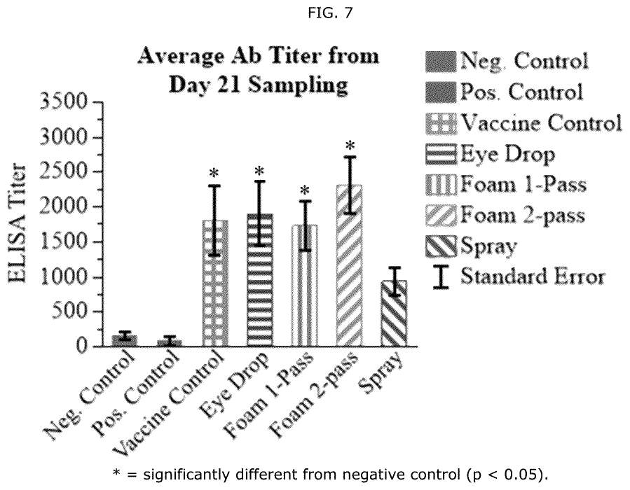

In order from highest to lowest, foam with 2-application vaccination had an average antibody titer of 2318±406, eyedrop vaccination had an average titer of 1899±460, the vaccine control group had an average titer of 1806±487, foam with 1-application vaccination had an average titer of 1730±361, spray vaccination had an average titer of 936±200, the negative control group had an average titer of 156±53, and the positive control group had an average titer of 87±67 (FIG. 7). The 2-application foam vaccination, eye drop vaccination, vaccine control group, and foam with 1-application vaccination were all significantly different from the negative and positive controls. Spray vaccination was not significantly different from the controls.

The manufacturer's instructions note that samples with titers >4000 are suspected of being infected. The ELISA reported that at the time of challenge, the vaccine control group and the one-application foam group had three birds with titers above this level, while the two-application foam group and the eyedrop group had 1 bird with titer above this level. The negative control, positive control, and spray vaccination groups had no birds above this level.

### Histopathological Lesion Scoring

Histopathological lesion scores of tracheal sections taken from the birds suggest that foam vaccination provides similar tracheal protection as eyedrop or spray vaccination. For the positive controls, 23 out of 23 samples had suspected active viral infections at the time of sampling and had an average lesion score of 6.22, that was significantly different from all the other groups. Foam 1-application vaccination had no samples that were suspected of having viral infection at the time of sampling and had an average score of 1.97 and was significantly different from the positive and negative control groups. Spray vaccination had 1 out of 34 samples that was suspected of having viral infection at the time of sampling and had an average score of 1.94 and was significantly different from the positive and negative control groups. The vaccine control had no samples that were suspected of having viral infection at the time of sampling and had an average score of 1.81 and was significantly different from the positive and negative control groups. The eyedrop vaccination group had no samples that were suspected of having viral infection at the time of sampling and had an average score of 1.29 and was significantly different from the positive control group. The foam 2-application vaccination group had no samples that were suspected of having viral infection at the time of sampling and had an average score of 1.16 and was significantly different from the positive control. The negative control group had no samples that were suspected of having viral infection at the time of sampling and an average score of 0.97 which was significantly different from the vaccine control, spray, foam 1-application, and positive control groups (Table 7).

TABLE 7
Average tracheal lesion scores and count of suspected virus
positive and virus negative samples for bird trial groups.

| Treatment | Suspected | Suspected | |
|---|---|---|---|
| Group | Positive | Negative | Average Lesion Score |
| Neg. Ctrl | 0 | 36 | 0.97 ± 0.17^{c} |
| Pos. Ctrl | 23 | 0 | 6.22 ± 0.26^{a} |
| Vac. Ctrl | 0 | 37 | 1.81 ± 0.17^{b} |
| Eyedrop | 0 | 38 | 1.29 ± 0.15^{bc} |
| Foam one- | 0 | 37 | 1.97 ± 0.19^{b} |
| application | | | |
| Foam two- | 0 | 32 | 1.16 ± 0.22^{bc} |
| application | | | |
| Spray | 1 | 33 | 1.94 ± 0.23^{b} |

Different letter subscripts indicate significant difference where p < 0.05. Averages are displayed as mean ± SE around the mean.

### Virus Isolation on Day 26

The virus isolation conducted on the tracheal swabs taken on day 26 of the trial suggest that foam vaccination elicits similar protection as eyedrop or spray vaccination. From most infected samples to least infected samples, the positive control group had 14 of 14 samples infected, spray vaccination had eight out of 33 samples infected, foam with 1-application vaccination had five out of 31 samples infected, eyedrop vaccination had three out of 35 samples infected, foam with 2-application had one out of 21 samples infected, and the vaccine control group had 0 out of 32 samples that were infected (Table 8).

TABLE 8
Counts of virus positive and virus negative
samples from day 26 virus isolation testing

| | Virus | Virus |
|---|---|---|
| Treatment | Positive | Negative |
| Pos. Ctrl | 14 | 0 |
| Vac. Ctrl | 0 | 32 |
| Eyedrop | 3 | 32 |
| Foam 1-application | 5 | 26 |
| Foam 2-application | 1 | 20 |
| Spray | 8 | 25 |

### Study Results and Conclusions

The results from this study suggest that the foam vaccination system successfully inoculated and protected chicks by utilizing foam as the vaccine vehicle in either one or two-pass application scenarios. The uniformity of application is reflected by the titers seen for the RT-PCR. The two-application had an average titer of 14942 ±1969 for RT-PCR while the 1-application application had an average titer of 891±2306, both of which are not significantly different from the spray vaccination titer of 11199±3607. The ELISA further confirmed this with the one-application application attaining an average ab titer of 1730±361 and the 2-application vaccination attaining an average ab titer of 2318±406 compared to spray vaccinations average titer of 936±200. The protection provided by the foam is seen in the tracheal lesion scores with 1-application application having an average score of 1.97±0.19 and 2-application application having an average lesion score of 1.16±0.22. These scores were not different from either the challenged eyedrop groups score (1.29±0.15) or the spray groups score (1.94±0.23). The induction of neutralizing immunity is also seen in both 1-application and 2-application applications with one-application application having only 5 out of 31 samples that had active viral infection and two-application application having only 1 out of 21 samples with an active viral infection. These were both better than the spray vaccination which had 8 out of 33 samples that showed signs of live, replicating virus.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A method for vaccinating one or more avians in need thereof, comprising administering to the one or more avians a stable foam in an amount effective for inducing a neutralizing immune response against an infectious pathogen in the one or more avians, wherein the foam comprises a gas and a liquid, and wherein the liquid comprises a vaccine and a foaming agent.

2. The method of claim 1, wherein the foam is ingestible.

3. The method of claim 2, further comprising ingesting the foam by the one or more avians.

4. The method of claim 1, further comprising contacting the foam with an eye of the one or more avians, whereby the vaccine enters the one or more avians via the eye.

5. The method of claim 1, wherein the one or more avians are one or more day-old chicks.

6. The method of claim 1, wherein the infectious pathogen causes an infectious disease selected from the group consisting of Marek's disease, coccidiosis, mycoplasma gallisepticum infections, infectious bursal disease, encephalomyelitis, fowlpox, laryngotracheitis, Newcastle disease, and infectious bronchitis.

7. The method of claim 1, wherein the foaming agent comprises a surfactant.

8. The method of claim 7, wherein the surfactant comprises one or more proteins.

9. The method of claim 1, wherein the foaming agent comprises a viscosity agent.

10. The method of claim 1, wherein the foaming agent comprises an aqueous solvent.

11. The method of claim 1, wherein the foam has an expansion rate of 20-40, a bubble size of 3-4 mm, a liquid drainage of 50-95% at 10 minutes, a deterioration rate of 95-99% at 5 minutes, or a combination thereof.

12. The method of claim 1, wherein the ratio of the liquid to the gas is from 1:20 to 1:200.

13. A method for preparing a stable vaccine foam, comprising:
(a) mixing a foaming agent and a vaccine capable of inducing a neutralizing immune response against an infectious pathogen in an avian in need thereof, whereby a liquid is formed;
(b) mixing the liquid with a gas, whereby a liquid gas mixture is formed; and
(c) forcing the liquid gas mixture through a filter, whereby a stable foam is formed.

14. The method of claim 13, wherein the foaming agent comprises a surfactant.

15. The method of claim 14, wherein the surfactant comprises one or more proteins.

16. The method of claim 13, wherein the foaming agent comprises a viscosity agent.

17. The method of claim 14, wherein the foaming agent comprises an aqueous solvent.

18. The method of claim 14, wherein the ratio of the liquid to the gas is from 1:20 to 1:200.

19. The method of claim 14, wherein the foam is ingestible.

20. The method of claim 14, wherein the avian is a day-old chick.
